(19) United States
(12) Patent Application Publication
(10) Pub. No.: US 2025/0001149 A1
(43) Pub. Date: Jan. 2, 2025

(54) SYSTEMS, DEVICES, AND METHODS INKJET MICRO-DOSING TECHNOLOGY TO DISPENSE A UNIFORM LAYER OF SKINCARE ACTIVES

(71) Applicant: L'Oreal, Paris (FR)

(72) Inventors: Gregoire CHARRAUD, Jersey City, NJ (US); Hyosik Dennis MIN, Palisades Park, NJ (US); Rafael FELICIANO, New Providence, NJ (US)

(73) Assignee: L'Oreal, Paris (FR)

(21) Appl. No.: 18/345,072

(22) Filed: Jun. 30, 2023

(51) Int. Cl. A61M 35/00; A45D 34/04

(52) U.S. Cl. CPC A61M 35/003; A45D 34/04; A45D 2034/002; A45D 2200/10; A61M 2205/13; A61M 2205/3327; A61M 2205/3379

(57) ABSTRACT

An applicator, including a body, an applicator surface having two or more nozzles configured to apply two or more formulas to skin, one or more reservoirs configured to hold the two or more formulas, a position sensor configured to map the skin as the applicator surface applies the two or more formulas, wherein the position sensor is configured to transmit position data, where the applicator is configured to adjust a dosage of each formula of the two or more formulas based on the position data.

### SUMMARY

In one aspect, disclosed herein is an applicator, including an applicator surface having two or more nozzles configured to apply two or more formulas to skin, one or more reservoirs configured to hold the two or more formulas, a position sensor configured to map the skin as the applicator surface applies the two or more formulas, where the position sensor is configured to transmit position data, and where the applicator is configured to adjust a dosage of each formula of the two or more formulas based on the position data.

In some embodiments, the two or more formulas comprise a first active formula and a second active formula. In some embodiments, each nozzle of the two or more nozzles is configured to dispense the first active formula or the second active formula. In some embodiments, the applicator surface is configured to dispense the first active formula and the second active formula in a two-dimensional matrix.

In some embodiments, the two or more formulas comprise a first active formula, a second active formula, and a third active formula. In some embodiments, the applicator surface is configured to dispense the first active formula, the second active formula, and the third active formula in a three-dimensional matrix.

In some embodiments, the applicator surface is configured to mix the two or more formulas on the skin after the two or more formulas are dispensed. In some embodiments, the applicator further comprises a condition sensor configured to detect a condition of the skin, wherein the condition sensor is configured to transmit condition data. In some embodiments, the condition sensor is a hydration sensor. In some embodiments, the applicator is configured to further adjust the dosage of each of the two or more formulas based on the condition data.

In another aspect, disclosed herein is a system for applying two or more formulas, the system including an applicator as described herein, and a dispensing device configured to couple to the applicator, where the dispensing device comprises a dispenser processor configured to direct the applicator to apply the two or more formulas.

In some embodiments, the two or more formulas comprise a first active formula and a second active formula. In some embodiments, each nozzle of the two or more nozzles is configured to dispense the first active formula or the second active formula. In some embodiments, the applicator surface is configured to dispense the first active formula and the second active formula in a two-dimensional matrix.

In some embodiments, the system further comprises a communication device communicatively coupled to the dispensing device. In some embodiments, the communication device is configured to visualize the skin, highlight one or more areas of the skin, where the one or more areas of the skin are areas on the skin where a user has not applied the two or more formulas, locations based on a user history, or a combination thereof, direct the user to move the applicator over the one or more areas, and store the user history, wherein the user history is each time, location, or a combination thereof that the user applies the two or more formulas.

In some embodiments, the applicator further includes a condition sensor configured to detect a condition of the skin, where the condition sensor is configured to transmit condition data. In some embodiments, the applicator is configured to further adjust the dosage of each of the two or more formulas based on the condition data.

In yet another aspect, described herein is a method of using the system disclosed herein, wherein the method includes moving the applicator across the skin, detecting a position of the applicator with the position sensor, determining an amount of each formula of the two or more formulas based on the position, and applying the amount of each formula to the skin.

In some embodiments, the method further includes determining an amount of a first active formula and an amount a second active formula of the two or more formulas, applying the first active formula and the second active formula in a ratio based on the position of the applicator, and adjusting the amount of the first active formula, the amount of the second active formula, or both in a two dimensional matrix, based on the position of the applicator.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A is an example applicator, in accordance with the present technology;

FIG. 1B is an example cross-sectional view of the applicator of FIG. 1A, in accordance with the present technology;

FIG. 1C is an example cross-sectional view of the applicator of FIG. 1A, in accordance with the present technology;

FIGS. 2A-2D are example top views of applicators, in accordance with the present technology;

FIG. 3 is an example two-dimensional matrix, in accordance with the present technology;

FIG. 4 is an example three-dimensional matrix, in accordance with the present technology;

FIG. 5 is an example system, in accordance with the present technology;

FIG. 6 is another example system in accordance with the present technology;

FIG. 7 is an example method of using a system, in accordance with the present technology; and

FIG. 8 is another example method of using a system, in accordance with the present technology.

### DETAILED DESCRIPTION

Disclosed herein are applicators, systems, and methods for applying two or more formulas to skin while continually adjusting the dosage of the two or more formulas as needed based on the position of the applicator, a condition of the skin, or a combination thereof. In some embodiments, the two or more formulas are two or more active ingredients. In some embodiments, the two or more formulas include a first active formula and a second active formula. In some embodiments, the applicator includes two or more nozzles configured to dispense the two or more formulas.

By monitoring the position of the applicator and/or a condition of the skin, the applicator may adjust the amount and/or concentration of each formula of the two or more formulas in a matrix, such as a two-dimensional matrix, or a three-dimensional matrix, in order to treat the condition or the particular area of skin the applicator is moving over.

In some embodiments, the two or more formulas are skin care formulas. In some embodiments, the skin care formulas include a moisturizer, a toner, an acne treatment, a wrinkle treatment, fine line treatment, a cosmetic, or a combination thereof. In some embodiments, the two or more formulas are two or more ingredients of a skin care formula. In some embodiments, the condition may include redness, acne, dryness, oiliness, and the like.

In this manner, the applicators, systems, and methods disclosed herein may dynamically adjust the composition of two or more formulas applied to the skin based on one or more conditions of the skin, or the location of the applicator, to deliver a personalized formula for each individual user.

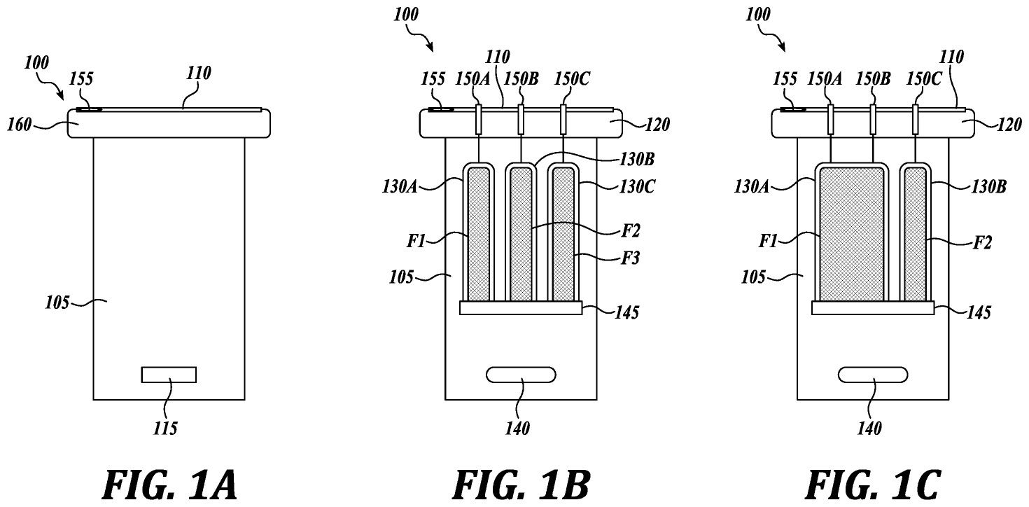

FIG. 1A is an example applicator, in accordance with the present technology. The applicator 100 may include a body 105, an applicator surface 110, a tag 115, an attachment 120, and one or more sensors 155.

In some embodiments, the applicator 100 includes a body 105. In some embodiments, the body 105 is configured to contain additional components and/or circuitry, as shown in FIGS. 1B and 1C.

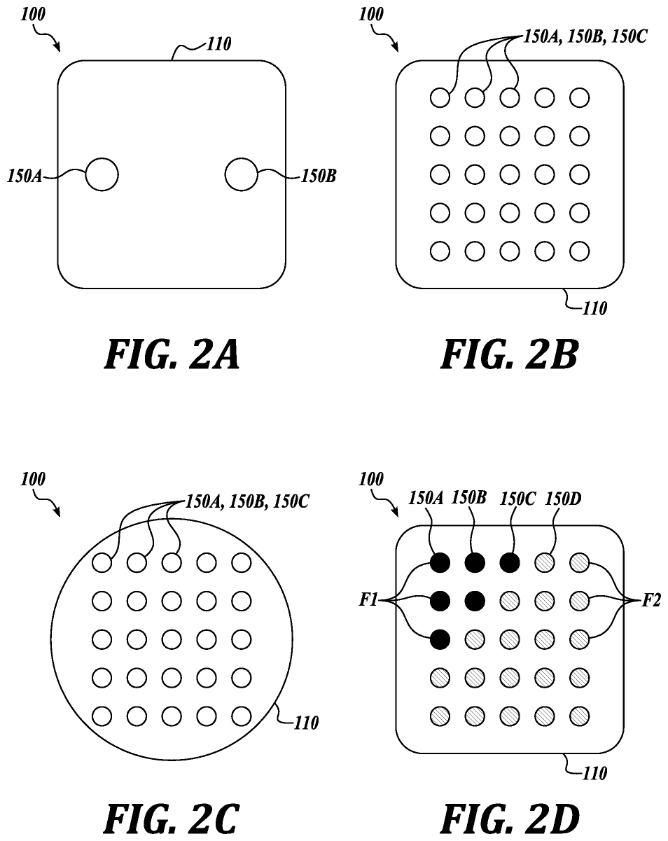

In some embodiments, the applicator includes a flat applicator surface 110. In operation, the applicator surface 110 may be moved across the skin. In some embodiments, the applicator surface includes one or more nozzles, as shown in FIGS. 2B-2C.

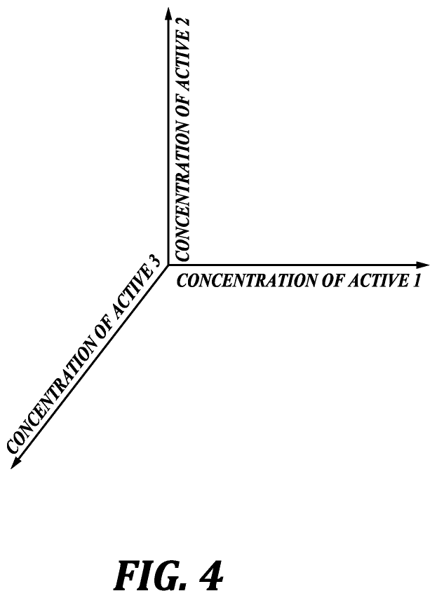

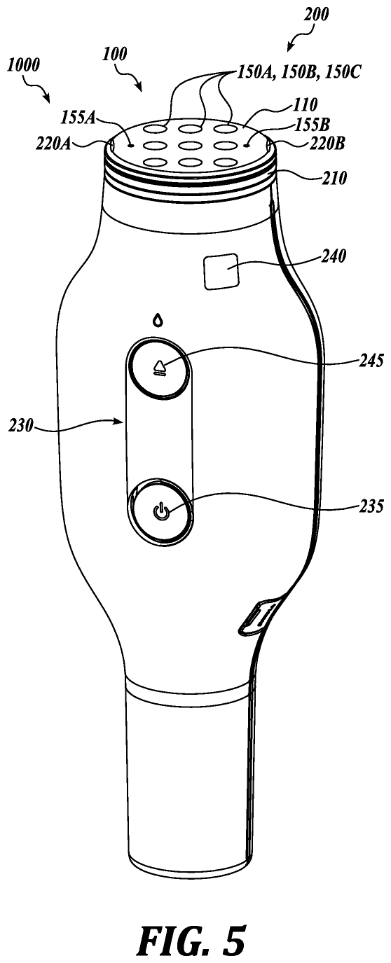

In some embodiments, the applicator 100 further includes a tag 115. In some embodiments, the tag 115 is a QR code, RFID tag, barcode, or the like. In some embodiments, the tag 115 communicates an identity of the applicator 100 or the one or more formulations to a dispensing device, such as shown in FIG. 4. In some embodiments, the tag 115 may be used to identify any number of things about the two or more formulas or applicator 100, including the amount of formula inside the applicator 100, the expiration date of the formula 140 inside the applicator 100, or when to replace the applicator 100. In some embodiments, the applicator 100 also includes an attachment 120 configured to secure the applicator 100 into a dispensing device, such as the dispensing device 200 in FIG. 4. While the attachment 120 is illustrated as a disk shaped to couple to a dispensing device, the attachment 120 may take any form capable of securing the applicator to a dispensing device including a threaded attachment, a magnet, or an attachment configured to snap into the dispensing device. In some embodiments, the attachment 120 is clear so that the dispensing device is visible through the attachment. In some embodiments, the attachment 120 may include any number of additional components, such as shown in detail in FIG. 5. In some embodiments, the applicator 100 is configured to apply cooling treatment.

In some embodiments, the applicator 100 includes one or more sensors 155. In some embodiments, the one or more sensors 155 include a position sensor and/or a condition sensor. In some embodiments, the one or more sensors 155 is a single sensor, configured to detect both a position and a condition on a user's skin. In some embodiments, the one or more sensors 155 are accelerometers, gyroscopes, or optical sensors, such as a camera. In such embodiments, the one or more sensors 155 are configured to determine where on a user's skin the applicator 100 is positioned. In some embodiments, the one or more sensors 155 are a condition sensor, such as a hydration sensor, a moisture sensor, a sensor configured to detect light reflecting of the skin, or the like. In such embodiments, the one or more sensors 155 may determine a condition of the skin as the applicator 100 moves over the skin. In some embodiments, the condition may include redness, acne, dryness, oiliness, and the like. In some embodiments, such as when the one or more sensors 155 are optical sensors, such as a camera, radar, and the like, the one or more sensors 155 may be configured to detect both a position and a condition of the user's skin, either simultaneously or sequentially. In some embodiments, the one or more sensors 155 are configured to transmit condition and/or position data to the applicator 100. In some embodiments, the one or more sensors are configured to transmit the condition and/or position data to a process, such as applicator processor 140 in FIGS. 1B-1C.

In operation, the applicator 100 can be placed inside a dispensing device (as shown in FIG. 4) and secured to the dispensing device with the attachment 120. The roller ball 110 can be rolled over a surface, such as a user's skin, to apply a formula.

FIG. 1B is an example cross-sectional view of the applicator 100 of FIG. 1A, in accordance with the present technology. In some embodiments, the applicator includes a body 105, an attachment 120, an applicator surface 110, and one or more formula reservoirs 130A, 130B, 130C configured to hold two or more formulas F1, F2, F3, a piston 145, one or more nozzles 150A, 150B, 150C, and an applicator processor 140.

In some embodiments, the one or more reservoirs 130A, 130B, 130C is located inside the body 105, and is configured to hold two or more formulas F1, F2, F3. In some embodiments, the two or more formulas F1, F2, F3 are skin care formulas. In some embodiments, the skin care formulas include a moisturizer, a toner, an acne treatment, a wrinkle treatment, fine line treatment, an eye cream, a cosmetic, or a combination thereof. In some embodiments, the two or more formulas F1, F2, F3 are one or more ingredients of a skin care formula. As the applicator surface 110 moves across the skin, the two or more formulas F1, F2, F3 from the one or more reservoirs 130 are applied to the skin. In some embodiments, the applicator surface is further configured to mix the two or more formulas F1, F2, F3 as it moves. In some embodiments, each formula reservoir 130 of the formula reservoirs 130A, 130B, 130C is configured to hold a distinct formula F of a plurality of formulas F1, F2, F3. In some embodiments, each formula reservoir 130 of the plurality of formula reservoirs 130A, 130B, 130C are fluidly coupled to a nozzle 150 of a plurality of nozzles 150A, 150B, 150C on the applicator surface 110. In such embodiments, each nozzle 150 is configured to dispense a different formula F1, F2, F3.

In some embodiments, the applicator 100 further includes a piston 145 configured to push the formula 140 towards one or more nozzles 150A, 150B, 150C as the formula is applied. In some embodiments, the piston 145 is directed by circuitry on a dispensing device or on the applicator itself to push the formula 140 towards the applicator surface.

In some embodiments, the applicator 100 includes an applicator processor 140. In some embodiments, the applicator processor 140 is configured to direct the piston 145 to push the formulas F1, F2, F3 towards the applicator surface. Further, in some embodiments, the applicator processor 140 is configured to receive position and/or condition data from the one or more sensors 150.

In some embodiments, the applicator surface is configured to dispense the first active formula F1, the second active formula F2, and the third active formula F3 in a three-dimensional matrix, as shown and described in FIG. 4. In some embodiments, the applicator surface 110 is configured to mix the two or more formulas on the skin after the two or more formulas are dispensed.

FIG. 1C is another example cross sectional view of the applicator of FIG. 2A, in accordance with the present technology. In some embodiments, the applicator includes a body 105, an attachment 120, an applicator surface 110, and one or more formula reservoirs 130A, 130B, configured to hold two or more formulas F1, F2 one or more nozzles 150A, 150B, 150C, and an applicator processor 140.

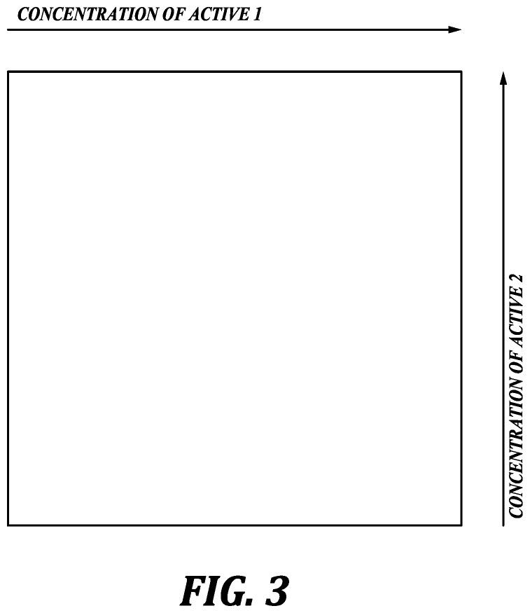

In some embodiments, the two or more formulas F1, F2 comprise a first active formula F1 and a second active formula F2. In some embodiments, each nozzle 150 of the two or more nozzles 150A, 150B, 150C is configured to dispense the first active formula F1 or the second active formula F2. In operation, the applicator surface 110 is configured to dispense the first active formula F1 and the second active formula F2 in a two-dimensional matrix, as shown and described in FIG. 3.

In some embodiments, a reservoir 130 (such as reservoir 130A) of the plurality of reservoirs 130A, 130B may be fluidly coupled to more than one nozzle 150 (such as nozzles 150A, 150B). In this manner, the applicator 100 may adjust the amount of the first active formula F1 and the second active formula F2 at a predetermined ratio. In some embodiments, the size of the reservoir 130 or the number of nozzles 150 fluidly coupled to the reservoir 130 may be determined by how much of the active formula F is needed. For example, the first reservoir 130A may include a moisturizer F1, while the second reservoir 130B may include an eye cream F2. As the eye cream would generally be needed in smaller quantities than a moisturizer, the eye cream may be contained in the smaller, second reservoir 130B.

In operation, the one or more sensors 155 may determine how the two or more formulas F1, F2 are dispensed. For example, in the case of a moisturizer F1 and an eye cream F2, the one or more sensors 155 may be configured to determine a position on the user's skin. As the applicator 100 moves towards the user's eyes, the applicator processor 140 directs the applicator 100 to apply the eye cream F1. Similarly, as the applicator 100 moves towards other portions of the user's skin, such as the forehead or cheeks, the applicator processor 140 directs the applicator to apply the moisturizer F2. When the applicator 100 transitions from under a user's eyes to the user's cheeks, the applicator processor 140 may direct the applicator to apply a mixture of eye cream F1 and moisturizer F2, adjusting the ratio depending on what position the applicator 100 is in, i.e., more eye cream when the applicator 100 is closer to the eye and less eye cream when the applicator 100 is farther from the eye.

In some embodiments, the one or more sensors 155 includes a condition sensor, such as a hydration sensor. In some embodiments, as the applicator 100 moves over the skin, the condition sensor detects a condition. In some embodiments, the condition determines the ratio of the active formulas F1, F2. For example, F1 may be a moisturizer and F2 may be hyaluronic acid. In such an example, when the condition sensor detects the condition of dryness, the amount or concentration of hyaluronic acid dispensed may be increased.

One skilled in the art will appreciate that in some embodiments, a single nozzle 150 is fluidly attached to more than one formula reservoir 130. In such embodiments, the single nozzle 150 may apply the first active formula F1, the second active formula F2, or both. In some embodiments, the decision of whether to apply the first active formula F1 or the second active formula F2 depends on the condition and/or position data.

In some embodiments, the applicator surface 110 is configured to mix the two or more formulas F1, F2 as the applicator 100 moves across the skin.

FIGS. 2A-2D are example top views of applicators 100, in accordance with the present technology. The applicator 100 may include an application surface 110 and two or more nozzles 150.

FIG. 2A is an applicator surface 110 having two nozzles 150A, 150B. In some embodiments, each nozzle 150 of the two nozzles 150A, 150B is configured to dispense a different formula, such as a first active formula and a second active formula. In some embodiments, the applicator directs the nozzles 150A, 150B to dispense the first active formula and the second active formula in a ratio. In some embodiments, the applicator further includes one or more sensors (as shown in FIGS. 1A-1C). In some embodiments, the ratio of the first active formula and the second active formula is determined based on a position of the applicator, a condition of skin, or a combination thereof.

FIG. 2B is an applicator surface 110 including a plurality of nozzles 150A, 150B, 150C (also referred to herein as an array of nozzles 150A, 150B, 150C). In some embodiments, the plurality of nozzles 150A, 150B, 150C are arranged in an array. While 25 nozzles are illustrated in FIG. 2B, it should be understood that any number of nozzles may make up the plurality of nozzles 150A, 150B, 150C. In some embodiments, each nozzle 150 of the plurality of nozzles 150A, 150B, 150C is fluidly coupled with an individual formula reservoir (such as shown in FIG. 1B). In some embodiments, multiple nozzles of the plurality of nozzles 150A, 150B, 150C are fluidly coupled to a single reservoir (such as shown in FIG. 1C). Further, in some embodiments, multiple nozzles of the plurality of nozzles 150A, 150B, 150C are fluidly coupled with two or more formula reservoirs. In such an embodiments, the multiple nozzles of the plurality of nozzles 150A, 150B, 150C may be configured to decide which formula of the two or more formulas will be dispensed based on condition and/or position data.

In some embodiments, the plurality of nozzles 150A, 150B, 150C is configured to dispense two or more formulas. In some embodiments, the plurality of nozzles 150A, 150B, 150C are configured to dispense a first active formula and a second active formula. In some embodiments, the plurality of nozzles 150A, 150B, 150C are configured to dispense a first active formula, a second active formula, and a third active formula. In some embodiments, the applicator 100 directs the plurality of nozzles 150A, 150B, 150C to dispense a single formula of the two or more formulas, or multiple formulas of the two or more formulas. In some embodiments, the applicator 100 directs the plurality of nozzles 150A, 150B, 150C to adjust a ratio or concentration of each formula of the two or more formulas depending on a position of the applicator, a condition of the skin, or both.

FIG. 2C is an example applicator surface 110 including one or more nozzles 150A, 150B, 150C. In some embodiments, the applicator surface 110 is round. It should be understood that the applicator surface 110 may take any shape. When the applicator surface 110 is round, it may be flush with a dispensing device (such as shown in FIG. 5).

FIG. 2D is an example applicator surface 110 in use, applying a first active formula F1 and a second active formula F2 from a plurality of nozzles 150A, 150B, 150C, 150D. In some embodiments, a first number of nozzles of the plurality of nozzles 150A, 150B, 150C, 150D are configured to dispense the first active formula F1, shown in FIG. 2D as 6 nozzles. Accordingly, a second number of nozzles of the plurality of nozzles 150A, 150B, 150C, 150D may dispense the second active formula F2, shown in FIG. 2D as 19 nozzles in FIG. 2D. It should be appreciated that any number of nozzles may be in the first number of nozzles and the second number of nozzles. In some embodiments, the first number of nozzles is equal to the second number of nozzles. Further, in some embodiments, both the first number of nozzles and the second number of nozzles may be configured to dispense both the first active formula F1 and the second active formula F2, both simultaneously and separately.

In this manner, in response to a position of the applicator 100, a condition of skin, or both, the applicator 100 may direct the plurality of nozzles 150A, 150B, 150C, 150D to dispense a specific concentration, amount, or ratio of the first active formula F1, the second active formula F2, or both.

FIG. 3 is an example two-dimensional matrix, in accordance with the present technology. In some embodiments, the applicator is configured to dispense a first active formula (active 1) and a second active formula (active 2) in a two-dimensional matrix.

As shown in FIG. 3, the two or more nozzles (such as shown in FIGS. 2A-2D) may be directed to adjust the concentration of the first active formula and the second active formula in a two-dimensional matrix. That is, the concentration of both the first active formula and the concentration of the second active formula may be adjusted independently to produce countless, personalized mixtures of the first active formula and the second active formula.

The applicator may direct the plurality of nozzles (such as shown in FIGS. 2A-2D) to adjust the number of nozzles dispensing each active formula, the amount of each formula of the two or more formulas being dispensed, or a combination thereof. In some embodiments, directing the plurality of nozzles includes directing a number of nozzles of the plurality of nozzles to apply no active formulas. In some embodiments, directing the plurality of nozzles includes switching a nozzle of the plurality of nozzles from applying the first active formula to applying the second active formula, or vice versa.

FIG. 4 is an example three-dimensional matrix, in accordance with the present technology. In some embodiments, the applicator is configured to dispense a first active formula (active 1), a second active formula (active 2), and a third active formula (active 3) in a three-dimensional matrix.

As shown in FIG. 4, the two or more nozzles (such as shown in FIGS. 2A-2D) may be directed to adjust the concentration of the first active formula, the second active formula, and the third active formula in a three-dimensional matrix. That is, the concentration of the first active formula, the concentration of the second active formula, and the concentration of the third active formula may be adjusted independently to produce countless, personalized mixtures of the first active formula, the second active formula, and the third active formula.

The applicator may direct the plurality of nozzles (such as shown in FIGS. 2A-2D) to adjust the number of nozzles dispensing each active formula, the amount of each active formula being dispensed, or a combination thereof. In some embodiments, directing the plurality of nozzles includes directing a number of nozzles of the plurality of nozzles to apply no active formulas. In some embodiments, directing the plurality of nozzles includes switching a nozzle of the plurality of nozzles from applying the first active formula to applying the second active formula or the third active formula, or vice versa.

FIG. 5 is an example system, in accordance with the present technology. In some embodiments, the applicator 100 can be attached to a dispensing device 200. In some embodiments, the applicator 100 includes two or more nozzles 150A, 150B, 150C, an applicator surface 110, and one or more sensors 155A, 155B. In some embodiments, the dispensing device includes one or more light sources 220A, 220B, and an actuator 230. In some embodiments, the one or more light sources 220A, 220B may be located on the dispensing device 100, the applicator 100, or both. In some embodiments, the either the dispensing device 200 or the applicator 100 includes one or more sensors 135.

In some embodiments, the dispensing device 200 includes an end 210. The end 210 may be configured to be seen through the attachment 120 on the applicator 100. In some embodiments, the end 210 includes one or more light sources 220A, 220B configured to administer light treatment to a surface while the formula is being applied.

In some embodiments, the one or more light sources 220A, 220B on either the dispensing device 200 or the applicator 100 are LEDs. In some embodiments, there are only two light sources 220A, 220B on the dispensing device. In some embodiments, a first light source 220A is configured to administer light therapy in a first wavelength. In some embodiments, a second light source 220B is configured to administer light therapy in a second wavelength. In some embodiments, the light therapy in the first wavelength and the light therapy in the second wavelength are administered simultaneously. In some embodiments, the light therapy and applying the formula happen simultaneously.

In some embodiments, the dispensing device 200 includes one or more actuators 230. While the actuator is illustrated as a button, in some embodiments, the actuator may be a switch, a capacitive touch type button, a dial, or the like. The actuator may be configured to begin the administration of light therapy, to apply the formula, or both. In some embodiments, the one or more actuators 230 include a power button 235 and an application button 245. In some embodiments, the power button 235 is configured to turn the dispensing device on or off, while the application button 245 is configured to administer one or more treatments described herein, including light treatment, radiation treatment, or application of two or more formulas. In some embodiments, the dispensing device 200 further includes one or more sensors 155A, 155B configured to detect a condition of the skin and/or a position of the applicator on the skin. In some embodiments, the one or more sensors 155A, 155B are located on the applicator 100, the dispensing device 200, or a combination thereof. In some embodiments, the dispensing device 200 further includes a device processor 240. In some embodiments, the dispenser processor 240 is configured to direct the applicator 100, the dispensing device 200, or both to administer one or more treatments in response to a condition detected by the one or more sensors 155A, 155B. In some embodiments, the dispensing device 200 also includes a contact-less chip reader (not pictured in FIG. 4) to read the tag 115 on the applicator 100.

In operation, a user may place an applicator 100 into the dispensing device 200. When the actuator 230 is actuated (such as by pressing the application button 245), the formula is applied, the light treatment is administered, or some combination of treatments is applied, concurrently or simultaneously.

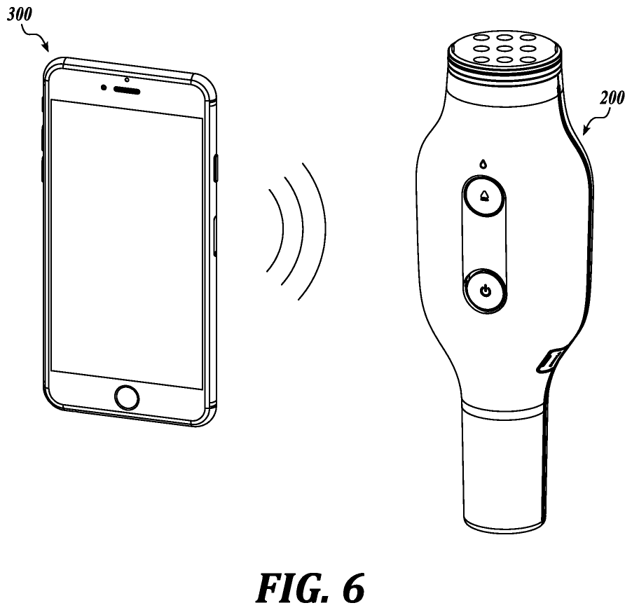

FIG. 6 is another example system in accordance with the present technology. The system, which implements an applicator 100 and a dispensing device 200 as described herein, further includes and a connected communication device 300. Optionally, the system may further include one or more external servers which are implemented as part of a cloud-computing environment.

The communication device 300 may be a personal computer (PC), a laptop computer, a PDA (Personal Digital Assistants), a smart phone, a tablet device, a UMPC (Ultra Mobile Personal Computer), a netbook, or a notebook type personal computer. In the below examples, the connected device 300 is assumed to be a smartphone, such as an Apple iPhone.

The communication device 300 is capable of performing wireless communication with the dispensing device 300 by way of a wireless communication interface circuitry on the dispensing device 200 or the applicator 100. However, communication device 300 is also capable of having a wired connection to the dispensing device 200 by way of a USB interface. Additionally, the applicator 100 and the dispensing device 200 may communicate with each other and the communication device 300 through an internet connection via an 802.11 wireless connection to a wireless internet access point, or a physical connection to the internet access point, such as through an Ethernet interface. Each connected communication device 300 is capable of performing wireless communication with other devices, such as through a Bluetooth connection or other wireless means as well.

The connected communication device 300 is configured to receive information from a user for use in generating a treatment plan, including one or more treatments, that may be used by the dispenser 200 to dispense two or more formulas, one or more light treatments, or a combination thereof.

In some embodiments, the communication device 300 has a related application on it, configured to aid a user in dispensing a formula and/or adjusting the concentration or amount of two or more formulas to create a personalized mixture. In some embodiments, the application is configured to apply an algorithm to a photo or video of a user to detect one or more conditions. In some embodiments, the conditions may include a dark circle, acne, pigmentation, rosacea, wrinkles, fine lines, or wounds. Additionally, in some embodiments, the application is configured to diagnose one or more skin conditions using an AI algorithm. In some embodiments, the communication device 300 receives the condition detected by the one or more sensors 155A, 155B and confirms or adjusts the detected condition with an AI algorithm. In some embodiments, the communication device 300 communicates the detected condition to a user of the system and proposes one or more treatments to be applied by the applicator 100.

In some embodiments, the communication device 300 may further sense one or more environmental conditions with one or more communication device sensors. In some embodiments, the environmental conditions may include temperature, humidity, UV, pollution, and the like. In some embodiments, the application can gather environmental data from other sources such as weather services. In some embodiments, the application can further recommend a formula, comprised of one or more skin ingredients, to the user based on the detected skin feature and/or environmental conditions.

In some embodiments, the user can set up a user profile on the application of the communication device 300. In some embodiments, setting up the user profile includes answering a user questionnaire. In some embodiments, the user questionnaire gives the user a series of inputs including past skin treatment, past use of the applicator, desired skin quality, or skin concern. In some embodiments, the application can solicit feedback from the user regarding their favorite or most effective formulation to help improve the algorithm.

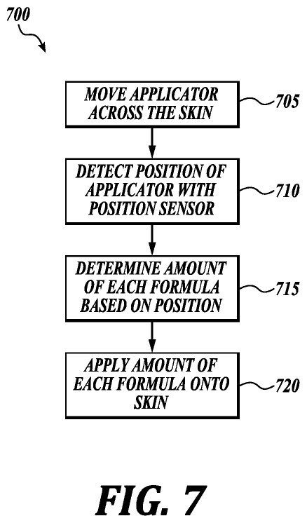

FIG. 7 is an example method 700 of using a system, in accordance with the present technology. It should be understood that the method 700 may be carried out with the applicator shown and described herein, and/or any of the systems shown and described.

In block 705, an applicator (such as applicator 100) is moved across the skin. In some embodiments, the applicator is moved across the skin with a user's hand.

In block 710, the position of the applicator is detected with a position sensor, such as position sensor 155 as described herein. In some embodiments, the position sensor is an accelerometer, a gyroscope, or an optical sensor, such as a camera. In some embodiments, the position sensor determines where on the skin the applicator is positioned.

In block 715, an amount of each formula of two or more formulas is determined based on the position of the applicator. In some embodiments, the amount of each formula is a concentration of each formula. In some embodiments, the two or more formulas are two formulas, i.e., a first active formula and a second active formula. In some embodiments, the two or more formulas are three formulas, i.e., a first active formula, a second active formula, and a third active formula.

In block 720, the amount of formula for each formula of the two or more formulas is applied onto the skin. In some embodiments, the amount of formula for each formula of the two or more formulas is determined as the applicator is moved across the skin and is adjusted dynamically.

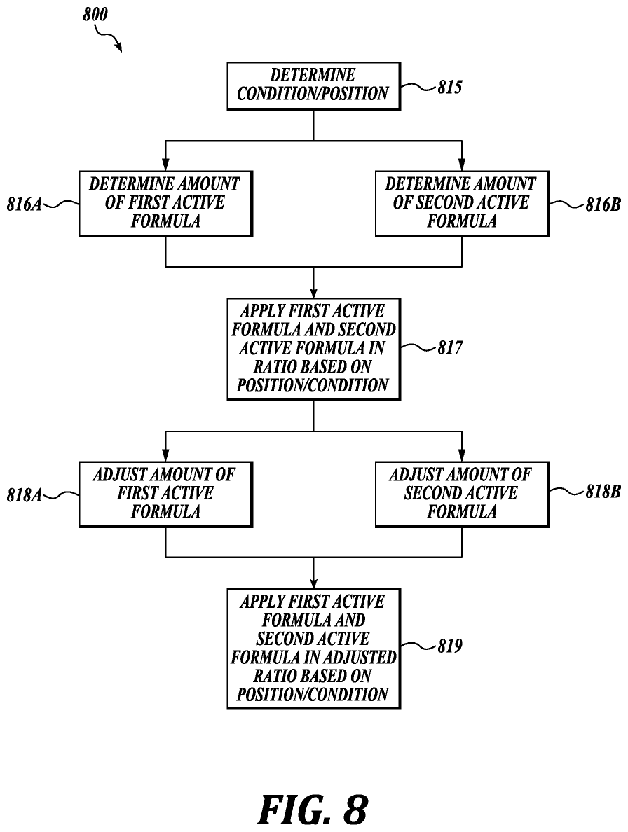

FIG. 8 is another example method 800 of using a system, in accordance with the present technology. In some embodiments, the method 800 may be carried out by an applicator (such as applicator 100) and/or a dispensing device (such as dispensing device 200). In some embodiments, the applicator includes an applicator surface (such as applicator surface 110), two or more nozzles (such as two or more nozzles 150), and one or more sensors (such as one or more sensors 155). In some embodiments, the one or more sensors include a position sensor and/or a condition sensor. It should be understood that method 800 may be performed in tandem with, or within method 700.

In block 815, a position of the applicator and/or a condition of the skin is determined. In some embodiments, as the applicator is moved over the skin, the applicator is configured to determine a position of the applicator and/or a condition of the skin under or around the applicator with one or more sensors. In some embodiments, the one or more sensors includes at least a position sensor and a condition sensors. Examples of position sensors include, but are not limited to, accelerometers, gyroscopes, optical sensors, and the like. Examples of condition sensors include, but are not limited to, hydration sensors, reflectance sensors, optical sensors, and the like. In some embodiments, the one or more sensors may be a single camera configured to detect both a position of the applicator and a condition of the skin.

In block 816A, an amount of a first active formula is determined. In some embodiments, the applicator is configured to apply two or more formulas. In some embodiments, the two or more formulas include a first active formula and a second active formula. One skilled in the art will appreciate that any number of active formulas may be applied by the applicator. In some embodiments, the first active formula amount or concentration is determined by the position, condition, or both. For example, in some embodiments, the amount/concentration of the first active formula is determined because the applicator is located on a cheek, nose, eyelid, forehead, or the like of the user. In some embodiments, the amount/concentration of the first active formula is determined by a condition, such as acne, redness, dryness, oiliness, or the like. For example, if the first active formula is a moisturizer, the concentration or amount may be increased when the skin is determined to be dry. Further, the concentration or amount may be reduced when the skin is determined to be red, indicating irritation.

In block 816B, an amount of a second active formula is determined. In some embodiments, the second active formula amount or concentration is determined by the position, condition, or both. For example, in some embodiments, the amount/concentration of the first second formula is determined because the applicator is located on a cheek, nose, eyelid, forehead, or the like of the user. In some embodiments, the amount/concentration of the first active formula is determined by a condition, such as acne, redness, dryness, oiliness, or the like. For example, if the second active formula was a concealer, the concentration or amount of the first active formula may be increased when the applicator is located at an undereye, a nose, or under an eyebrow. Further, the amount or concentration of the first active formula may be increased when the condition sensor detects acne on the skin. Similarly, the amount or concentration of the second active formula may be reduced, or not even applied, when the condition sensor does not detect acne or redness, or when the position sensor determines the applicator is located somewhere concealer is not generally applied, such as the cheeks. As shown in method 800, the adjustment of the amount or concentration of both the first active formula and the second active formula is independent. In this manner, the first active formula and the second active formula can be adjusted based on separate criteria, i.e., position or condition. Further, the same position and condition measurements may determine the amount or concentration of each active formula differently based on the identity of each active formula of the two or more formulas.

In block 817, the first active formula and the second active formula are applied in a ratio based on their individual amount/concentration determinations in blocks 816A and 816B.

In block 818A, the amount of the first active formula is adjusted. As the applicator moves across the skin, the position sensor and/or the condition sensor continue to measure the position/condition and update the amount of the first active formula to be applied. In this way, the applicator may adjust the amount of the first active formula in real time as the applicator moves across the skin.

In block 818A, the amount of the second active formula is adjusted. As the applicator moves across the skin, the position sensor and/or the condition sensor continue to measure the position/condition and update the amount of the second active formula to be applied. In this way, the applicator may adjust the amount of the second active formula in real time as the applicator moves across the skin.

In block 819, the first active formula and the second active formula are applied in an adjusted ratio based on the position and/or condition.

It should be understood that methods 700 and 800 should be interpreted as merely representative. In some embodiments, process blocks of methods 700 and 800 may be performed simultaneously, sequentially, in a different order, or even omitted, without departing from the scope of this disclosure.

The present application may reference quantities and numbers. Unless specifically stated, such quantities and numbers are not to be considered restrictive, but representative of the possible quantities or numbers associated with the present application. Also, in this regard, the present application may use the term “plurality” to reference a quantity or number. In this regard, the term “plurality” is meant to be any number that is more than one, for example, two, three, four, five, etc. The terms “about,” “approximately,” “near,” etc., mean plus or minus 5% of the stated value. For the purposes of the present disclosure, the phrase “at least one of A, B, and C,” for example, means (A), (B), (C), (A and B), (A and C), (B and C), or (A, B, and C), including all further possible permutations when greater than three elements are listed.

Embodiments disclosed herein may utilize circuitry in order to implement technologies and methodologies described herein, operatively connect two or more components, generate information, determine operation conditions, control an appliance, device, or method, and/or the like. Circuitry of any type can be used. In an embodiment, circuitry includes, among other things, one or more computing devices such as a processor (e.g., a microprocessor), a central processing unit (CPU), a digital signal processor (DSP), an application-specific integrated circuit (ASIC), a field-programmable gate array (FPGA), or the like, or any combinations thereof, and can include discrete digital or analog circuit elements or electronics, or combinations thereof.

An embodiment includes one or more data stores that, for example, store instructions or data. Non-limiting examples of one or more data stores include volatile memory (e.g., Random Access memory (RAM), Dynamic Random Access memory (DRAM), or the like), non-volatile memory (e.g., Read-Only memory (ROM), Electrically Erasable Programmable Read-Only memory (EEPROM), Compact Disc Read-Only memory (CD-ROM), or the like), persistent memory, or the like. Further non-limiting examples of one or more data stores include Erasable Programmable Read-Only memory (EPROM), flash memory, or the like. The one or more data stores can be connected to, for example, one or more computing devices by one or more instructions, data, or power buses.

In an embodiment, circuitry includes a computer-readable media drive or memory slot configured to accept signal-bearing medium (e.g., computer-readable memory media, computer-readable recording media, or the like). In an embodiment, a program for causing a system to execute any of the disclosed methods can be stored on, for example, a computer-readable recording medium (CRMM), a signal-bearing medium, or the like. Non-limiting examples of signal-bearing media include a recordable type medium such as any form of flash memory, magnetic tape, floppy disk, a hard disk drive, a Compact Disc (CD), a Digital Video Disk (DVD), Blu-Ray Disc, a digital tape, a computer memory, or the like, as well as transmission type medium such as a digital and/or an analog communication medium (e.g., a fiber optic cable, a waveguide, a wired communications link, a wireless communication link (e.g., transmitter, receiver, transceiver, transmission logic, reception logic, etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

The detailed description set forth above in connection with the appended drawings, where like numerals reference like elements, are intended as a description of various embodiments of the present disclosure and are not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed. Similarly, any steps described herein may be interchangeable with other steps, or combinations of steps, in order to achieve the same or substantially similar result. Generally, the embodiments disclosed herein are non-limiting, and the inventors contemplate that other embodiments within the scope of this disclosure may include structures and functionalities from more than one specific embodiment shown in the figures and described in the specification.

In the foregoing description, specific details are set forth to provide a thorough understanding of exemplary embodiments of the present disclosure. It will be apparent to one skilled in the art, however, that the embodiments disclosed herein may be practiced without embodying all the specific details. In some instances, well-known process steps have not been described in detail in order not to unnecessarily obscure various aspects of the present disclosure. Further, it will be appreciated that embodiments of the present disclosure may employ any combination of features described herein.

The present application may include references to directions, such as “vertical,” “horizontal,” “front,” “rear,” “left,” “right,” “top,” and “bottom,” etc. These references, and other similar references in the present application, are intended to assist in helping describe and understand the particular embodiment (such as when the embodiment is positioned for use) and are not intended to limit the present disclosure to these directions or locations.

The present application may also reference quantities and numbers. Unless specifically stated, such quantities and numbers are not to be considered restrictive, but exemplary of the possible quantities or numbers associated with the present application. Also, in this regard, the present application may use the term “plurality” to reference a quantity or number. In this regard, the term “plurality” is meant to be any number that is more than one, for example, two, three, four, five, etc. The term “about,” “approximately,” etc., means plus or minus 5% of the stated value. The term “based upon” means “based at least partially upon.”

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

While illustrative embodiments have been illustrated and described, it will be appreciated that various changes can be made therein without departing from the spirit and scope of the invention.

1. An applicator, comprising:
an applicator surface having two or more nozzles configured to apply two or more formulas to skin;
one or more reservoirs configured to hold the two or more formulas;
a position sensor configured to map the skin as the applicator surface applies the two or more formulas, wherein the position sensor is configured to transmit position data, and
wherein the applicator is configured to adjust a dosage of each formula of the two or more formulas based on the position data.

2. The applicator of claim 1, wherein the two or more formulas comprise a first active formula and a second active formula.

3. The applicator of claim 2, wherein each nozzle of the two or more nozzles is configured to dispense the first active formula or the second active formula.

4. The applicator of claim 2, wherein the applicator surface is configured to dispense the first active formula and the second active formula in a two-dimensional matrix.

5. The applicator of claim 1, wherein the two or more formulas comprise a first active formula, a second active formula, and a third active formula.

6. The applicator of claim 5, wherein the applicator surface is configured to dispense the first active formula, the second active formula, and the third active formula in a three-dimensional matrix.

7. The applicator of claim 1, wherein the applicator surface is configured to mix the two or more formulas on the skin after the two or more formulas are dispensed.

8. The applicator of claim 1, wherein the applicator further comprises a condition sensor configured to detect a condition of the skin, wherein the condition sensor is configured to transmit condition data.

9. The applicator of claim 8, wherein the condition sensor is a hydration sensor.

10. The applicator of claim 8, wherein the applicator is configured to further adjust the dosage of each of the two or more formulas based on the condition data.

11. A system for applying two or more formulas, the system comprising:
an applicator of claim 1, and
a dispensing device configured to couple to the applicator, wherein the dispensing device comprises a processor configured to direct the applicator to apply the two or more formulas.

12. The system of claim 11, wherein the two or more formulas comprise a first active formula and a second active formula.

13. The system of claim 12, wherein each nozzle of the two or more nozzles is configured to dispense the first active formula or the second active formula.

14. The system of claim 12, wherein the applicator surface is configured to dispense the first active formula and the second active formula in a two-dimensional matrix.

15. The system of claim 11, wherein the system further comprises a communication device communicatively coupled to the dispensing device.

16. The system of claim 15, wherein the communication device is configured to:
visualize the skin;
highlight one or more areas of the skin, wherein the one or more areas of the skin are areas on the skin where a user has not applied the two or more formulas, locations based on a user history, or a combination thereof;
direct the user to move the applicator over the one or more areas; and
store the user history, wherein the user history is each time, location, or a combination thereof that the user applies the two or more formulas.

17. The system of claim 11, wherein the applicator further comprises a condition sensor configured to detect a condition of the skin, wherein the condition sensor is configured to transmit condition data.

18. The system of claim 17, wherein the applicator is configured to further adjust the dosage of each of the two or more formulas based on the condition data.

19. A method of using the system of claim 11, wherein the method comprises:
moving the applicator across the skin;
detecting a position of the applicator with the position sensor;
determining an amount of each formula of the two or more formulas based on the position; and
applying the amount of each formula to the skin.

20. The method of claim 19, wherein the method further comprises:
determining an amount of a first active formula and an amount a second active formula of the two or more formulas;
applying the first active formula and the second active formula in a ratio based on the position of the applicator; and
adjusting the amount of the first active formula, the amount of the second active formula, or both in a two-dimensional matrix, based on the position of the applicator.
